(19) United States
(12) Patent Application Publication
(10) Pub. No.: US 2026/0026870 A1
(43) Pub. Date: Jan. 29, 2026

(54) CONNECTION ASSEMBLY, METHOD FOR PRODUCING A CONNECTION ASSEMBLY AND SURGICAL INSTRUMENT

(71) Applicant: KARL STORZ SE & Co. KG, Tuttlingen (DE)

(72) Inventors: Dominik LÄNGLE, Mühlheim (DE); Robin MERZ, Tuttlingen (DE)

(21) Appl. No.: 18/998,256

(22) PCT Filed: Jul. 25, 2023
(86) PCT No.: PCT/EP2023/070501

§ 371 Date: Jan. 24, 2025

(30) Foreign Application Priority Data
Jul. 26, 2022 (DE) 10 2022 118 628.8

(51) Int. Cl. A61B 18/14; A61B 17/00; A61B 17/29; A61B 18/00

(52) U.S. Cl. CPC A61B 18/1445; A61B 17/29; A61B 18/1482; A61B 2017/00464; A61B 2017/00486; A61B 2017/2929; A61B 2018/00178

(57) ABSTRACT

A connection assembly for a surgical instrument or in a surgical instrument, includes an instrument base that is configured to control and/or operate a high-frequency tool of the surgical instrument, a high-frequency connection that has a connection element with a blocking section and an orientation section for the predetermined orientation of a connection axis of the high-frequency connection within the instrument base, and a rotary wheel assembly having a coupling section for coupling to the blocking section of the high-frequency connection, such that a rotary wheel axis of the rotary wheel assembly can be orientated at a predetermined angle relative to the connection axis via the coupling process. A method produces a connection assembly of this type and a surgical instrument includes such a connection assembly of this type.

### CROSS REFERENCE TO RELATED APPLICATIONS

This application is a United States National Phase Application of International Application PCT/EP2023/070501, filed Jul. 25, 2023, and claims the benefit of priority under 35 U.S.C. § 119 of German Application 10 2022 118 628.8, filed Jul. 26, 2022, the entire contents of which are incorporated herein by reference.

### TECHNICAL FIELD

The present invention relates to a connection assembly for a surgical instrument. The invention also relates to a method for producing a connection assembly of this type and to a corresponding surgical instrument.

### TECHNICAL BACKGROUND

Surgical instruments are used for different applications. For example, they can be designed as minimally invasive medical instruments for high-frequency surgery and comprise a base, for example in the form of a handling device, at the proximal end, a long and usually thin shaft extending from the proximal end to a distal end of the instrument, and a high-frequency accessory, i.e. a tool designed for HF treatment or another operative device for gripping, squeezing, coagulating, cutting, punching or for other applications at the distal end of the instrument. One or more transmission devices run in the shaft for transmitting a force and/or a torque from the handling device at the proximal end to the operative device at the distal end. Furthermore, for the electrosurgical function, especially in monopolar or bipolar electrosurgical minimally invasive medical instruments, the transmission device is also involved in a transmission of electrical power from the proximal end to the distal end.

The instruments can often be disassembled into individual components, such that different tools, different shafts and other connection elements can be combined. Advantageously, a system with different and therefore versatile application possibilities can thus be provided.

High-quality minimally invasive medical instruments are also usually designed to be reusable. In order to simplify cleaning after use, to allow replacement of a defective component and/or alternative use of different components, a high-quality minimally invasive medical instrument can advantageously be disassembled.

On the one hand, in the case of a disassemblable medical instrument in which the transmission device is also involved in the transmission of electrical power, a mechanically separable and safely re-establishable electrical contact with the transmission device should be possible, particularly at the proximal end of the instrument.

For example, the publication DE 10 2017 124 775 A1 describes a minimally invasive medical instrument with an instrument base and an accessory shaft that can be inserted into the instrument base and has a transmission device for transmitting electrical power and for transmitting a force and/or a torque from a proximal position to a distal position. A contact device is arranged in the instrument base and is connected to a plug contact on the side facing away from the accessory shaft.

A disadvantage of such instruments is that the plug contact, for example a high-frequency connections, is permanently installed in the instrument base, i.e. in the handle element.

Another disadvantage of an HF connection element is that it requires a complex sleeve system with internal cables to ensure the required safety distances and sufficient insulation. During assembly, the cables must therefore be connected in a complex manner. Care must be taken to ensure that no increased contact resistance occurs at the transition points between the plug sleeves and the relevant cable. Correct installation is therefore time-consuming and requires sufficient specialist knowledge.

### SUMMARY OF THE INVENTION

Against this background, the present invention is based on the object of providing an improved connection assembly for a surgical instrument.

According to the invention, this object is achieved by means of a connection assembly having features according to the invention, by means of a method having features according to the invention, and by means of a surgical instrument having features according to the invention.

Accordingly, the following are provided:
A connection assembly for a surgical instrument or in a surgical instrument, comprising an instrument base that is configured to control and/or operate a high-frequency tool of the surgical instrument, a high-frequency connection that has a connection element with a blocking section and an orientation section for the predetermined orientation of a connection axis of the high-frequency connection within the instrument base, and a rotary wheel assembly having a coupling section for coupling to the blocking section of the high-frequency connection, such that a rotary wheel axis of the rotary wheel assembly can be orientated at a predetermined angle relative to the connection axis via the coupling process.
A method for producing a connection assembly for a surgical instrument, in particular a connection assembly, comprising the steps of: attaching a rotary wheel assembly to a shaft opening of an instrument base, wherein a coupling section of the rotary wheel assembly is inserted into the instrument base, then sliding a pre-assembled high-frequency connection into a connection slot of the instrument base, then establishing contact between the coupling section and a blocking section of the high-frequency connection, and then completely inserting and fastening the coupling section in the instrument base for coupling to the blocking section.
A surgical instrument comprising an accessory, in particular a high-frequency tool, for example a high-frequency forceps tool, and a connection assembly and/or assembled by a method wherein an accessory shaft is mechanically coupled to the rotary wheel assembly, and a high-frequency transmission element of the accessory, in particular a pull rod guided in the accessory shaft, is contacted with the high-frequency connection.

The finding underlying the present invention is that an orientation of the HF connection with respect to the rotary wheel assembly can be achieved when the rotary wheel assembly contacts the blocking section, in particular inside the instrument base.

The idea underlying the present invention is to orient the rotary wheel axis of the rotary wheel assembly at a predetermined angle to the connection axis via the coupling process itself by constructing the high-frequency connection (HF connection) with a blocking section and an orientation section. In this way, a modular design with pre-assembled elements can provide an assembly-friendly and technically optimized connection assembly.

A connection element is to be understood in particular as a structural element which, on the one hand, can be contacted with the rotary wheel assembly, in particular with a coupling section of the rotary wheel assembly, and, on the other hand, allows a predetermined orientation of the HF connection in the instrument base and ensures this in the assembled state. According to one embodiment, the connection element can, for example, have two sections angled towards each other, wherein one section is configured as a blocking section and the other section as an orientation section. The two sections angled towards each other allow the HF connection to be oriented in the instrument base and the HF connection to be oriented relative to the rotary wheel assembly. The blocking section and the orientation section thus allow the orientation of the rotary wheel axis with respect to the connection axis.

The design of the high-frequency connection according to the invention allows a surgical instrument for example to be modularly disassembled into individual components, such as a handle, the HF connection, an outer shaft and a working insert.

An instrument base suitable for the connection assembly can have different designs and, for example, comprise a handle element or a robot coupling. In particular, the instrument base is configured as a handle. The instrument base has at least one connection slot for inserting the HF connection. Furthermore, an accessory shaft or the like can be arranged on the instrument base, in particular in a shaft opening.

A connection slot is understood in particular to be a receptacle for the HF connection. The orientation of the connection slot in combination with the design of the connection element can define the orientation of the connection axis. In particular, the orientation of the connection slot and the connection axis can be the same. Likewise, the orientation of the rotary wheel axis can be defined by the orientation of the shaft opening.

The HF connection is configured as a pre-assembled insert module for modular installation in and removal from an instrument base. In this way, for an electrosurgical instrument, a modular system can be provided which is characterized by the highest possible number of pre-assembled components. In particular, this allows for easy exchange and/or switching between a monopolar and a bipolar design of the surgical instrument. Furthermore, such an HF connection can ensure a predetermined orientation and also contacting of the HF connection and a shaft connecting element with the accessory shaft within the instrument base.

Advantageous embodiments and developments are shown in the further dependent claims and in the description with reference to the figures in the drawing.

According to one embodiment, the blocking section can form a receptacle for the coupling section, which receptacle is concentric with the rotary wheel axis. This is achieved in particular by the blocking section being formed at an angle to the orientation section. While the orientation section substantially defines the orientation of the HF connection, the blocking section is substantially arranged on the rotary wheel axis of the rotary wheel assembly. This allows a fixedly predefined angle to be achieved between the rotary wheel axis and the connection axis during assembly and when the HF connection and the rotary wheel assembly are installed in the instrument base.

According to an advantageous embodiment, the connection element can have, in the region of the blocking section, a through-opening which is configured to guide a transmission element of a high-frequency tool, which transmission element can be passed through the rotary wheel assembly. In particular, the transmission element is a pull rod. The through-opening serves to guide and/or electrically contact the pull rod guided in an accessory shaft. The opening is advantageously larger than the largest diameter of a pull rod head of the pull rod element. The pull rod element can be passed through the through-opening and hooked onto an actuating element. In the case of a hand-operated surgical instrument, the actuating element may be a thumb ring element that can be movably mounted on the instrument base to actuate the pull rod.

According to one embodiment, the instrument base can be configured as a connecting device, for example to a manual guide element configured as a handle, to a manipulator coupling or to a robot holder. In particular, the instrument base is configured as a handle with an actuating element for manual actuation. The actuating element can have a finger ring and a thumb ring, wherein the thumb ring is coupled to the transmission element, in particular to the pull rod head of the pull rod. These can be used to control the accessory which is coupled to the transmission element, and which is preferably configured as a loop, clamp or scissors, by means of the handle. In a further embodiment, the instrument base can be configured as a manipulator coupling or as a robot holder for actuating the accessory coupled thereto via the accessory shaft.

According to a particularly preferred embodiment, the connection element can have, in the region of the blocking section, a protective geometry for protecting a high-frequency contact assembly, which is configured in particular to protect the at least one high-frequency contact element from damage when inserting the high-frequency tool into the instrument base. When inserting the HF tool into the instrument base, the pull rod head in particular comes into contact with the blocking section. The protective geometry ensures that the HF contacts present on the HF contact assembly are not damaged by the pull rod being brought closer or by contact with the pull rod.

According to an advantageous embodiment, the blocking section can have a blocking geometry which is configured for rotationally secure engagement with a coupling section of the rotary wheel assembly. The shape and dimensions of the blocking geometry can be adapted to the coupling section in such a way that a fixation between the HF connection and the rotary wheel assembly can be achieved. For example, the blocking geometry may be adapted to the geometry of a shaft connecting element that is slid in the rotary wheel assembly and coupled to the accessory shaft. A rotationally secure engagement can be achieved in particular by clamping and/or by latching.

According to a further development, the protective geometry and the blocking geometry can be formed integrally with each other. This means in particular that the protective geometry and the blocking geometry can be formed by one and the same component or the same components. For example, it can be an injection-molded part, preferably made of a plastic suitable for surgical use. Of course, additive manufacturing would also be conceivable in other embodiments. This means that the HF connection can be configured to be functionally integrated and yet space-saving, and can be manufactured easily and in large quantities.

According to one embodiment, the blocking geometry and/or the protective geometry can have at least one orientation projection, in particular two or more orientation projections, wherein the at least one orientation projection and the high-frequency contact assembly are arranged adjacent to one another. As a result, the HF contact assembly is arranged next to at least one orientation projection and projects beyond it in the longitudinal direction of the rotary wheel axis. This means that when the accessory shaft is inserted into the instrument base, it contacts the at least one orientation projection when contact is made with the HF contact assembly. In this way, a predetermined permissible elastic deformation of the HF contact assembly is possible without it being damaged by excessive deformation.

According to an advantageous embodiment, the high-frequency contact assembly can have two or more high-frequency contact elements pre-assembled on the blocking section, wherein the orientation projections and the high-frequency contact elements are arranged next to one another, preferably alternately, on the blocking section, such that the contact elements are each arranged in a gap between the orientation projections. This allows the HF contact elements to be correctly oriented in a pre-assembled HF connection on the one hand and, on the other hand, to be arranged in the gaps so that they are protected from excessive deformation.

Due to the arrangement in the gaps, even small tolerances can be correctly compensated for during assembly, since the freedom of movement of the HF contact elements is limited by the size of the gaps.

According to a further development, for the rotational orientation of the connection element and the rotary wheel assembly in the instrument base, at least one engagement element can be arranged on the connection element, in particular at a transition from the blocking section to the orientation section, and can be contacted with a corresponding coupling element of the rotary wheel assembly and/or the instrument base. This allows the HF connection to be oriented with respect to the rotary wheel assembly by engaging, clamping and/or latching.

According to a further development of the method, for fastening, the coupling section can be screwed into the instrument base, wherein the attachment of the rotary wheel assembly comprises securing the position by partial screwing-in. This advantageously ensures that the angle between the rotary wheel axis and the connection axis is always constant. This is important for correct use and proper functioning of the surgical instrument, as it prevents incorrect positioning or a change in the angle, which could lead to contact problems during power transmission, for example, and also prevents the pull rod from dragging or becoming blocked.

In order to fix the angle between the two axes, the rotary wheel assembly is advantageously first partially, in particular minimally, screwed into the instrument base during assembly. The HF connection is then slid into the instrument base on a side facing away from the accessory shaft. The rotary wheel assembly is then completely screwed in. In particular, the blocking section with the blocking geometry allows the two elements to be oriented exactly in relation to each other. The blocking geometry therefore preferably has a concentric guide between the HF connection and the rotary wheel assembly. Furthermore, a rotational orientation is preferably carried out, in particular via an engagement element.

According to a further embodiment of the method, the insertion of the coupling section into the shaft opening and the sliding of the high-frequency connection into the connection slot can be carried out from different directions, such that the coupling of the coupling section to the blocking section comprises joining without visibility. The contact between the HF connection of the rotary wheel assembly is therefore made inside the instrument base and can advantageously be made without the fitter having a direct view, due to the shape of the blocking section and of the orientation section.

According to a further embodiment of the method, the connection between the connection element and the coupling element of the rotary wheel assembly is configured as a plug-and-play connection, wherein the surgical instrument can be used directly after the connection assembly has been made. In particular, plug-and-play means that the surgical instrument can be used immediately after plugging in the HF connection and fixing it with the rotary wheel assembly. This allows the surgical instrument to be flexibly adapted to different requirements easily and without special knowledge, since the HF connection or the rotary wheel assembly and/or the accessory shaft with the tool can be exchanged on site and without the need for tools.

The above embodiments and developments can be combined with each other as desired, if appropriate. Further possible embodiments, developments, and implementations of the invention also include combinations, which are not explicitly mentioned, of features of the invention described above or below with respect to the exemplary embodiments. In particular, a person skilled in the art will also add individual aspects as improvements or additions to the particular basic form of the present invention.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

The present invention is explained in greater detail below with reference to the exemplary embodiments shown in the schematic figures of the drawings.

In the drawings:

FIG. 1 is a perspective view showing an embodiment of a shaft connecting element of a connecting device;

FIG. 2 is a side view showing a further embodiment of a shaft connecting element of a connecting device;

FIG. 3 is a sectional view showing an embodiment of a receiving device;

FIG. 4 is a sectional view showing the receiving device from FIG. 3 with the shaft connecting element slid in;

FIG. 5 is a sectional view showing the receiving device from FIG. 3 with a slid-in shaft connecting element and pull rod;

FIG. 6 is a sectional view showing a connecting device with non-matching elements;

FIG. 7 is a sectional view showing a connecting device with non-matching elements;

FIG. 8 is a sectional view showing a connecting device with non-matching elements;

FIG. 9 is a side and partial sectional view showing an embodiment of a connection assembly;

FIG. 10 is a perspective and partial sectional view of the embodiment of FIG. 9;

FIG. 11 is a perspective showing an embodiment of a connection element;

FIG. 12 is a further perspective view of the embodiment of FIG. 11;

FIG. 13 is a non-exploded and exploded view showing an embodiment of an instrument base with high-frequency connection;

FIG. 14 is a detailed view of the embodiment according to FIG. 13 with a detailed view of an embodiment of the high-frequency connection;

FIG. 15 is a sectional view showing an embodiment of a coupling of the pull rod head with the handle element;

FIG. 16 is a perspective detailed e view showing an embodiment of a connecting device;

FIG. 17 is a side view showing an embodiment of a surgical instrument;

FIG. 18 is a perspective detailed view showing a further embodiment of a connecting device;

FIG. 19 is a sectional view showing an embodiment of a shaft connecting element arranged on an accessory shaft.

The accompanying figures of the drawing are intended to provide a further understanding of the embodiments of the invention. They illustrate embodiments and, in conjunction with the description, serve to explain principles and concepts of the invention. Other embodiments and many of the advantages mentioned are shown in the drawings. The elements in the drawings are not necessarily shown to scale.

In the figures in the drawing, like, functionally like and identically acting elements, features and components are each provided with the same reference signs, unless otherwise specified.

### DESCRIPTION OF EXEMPLARY EMBODIMENTS

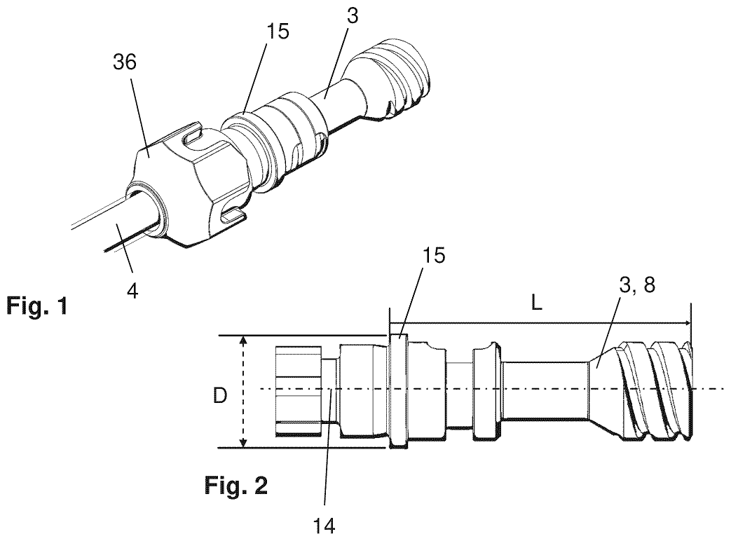

Referring to the drawings, FIG. 1 shows an embodiment of a shaft connecting element 3 of a connecting device 1. The shaft connecting element 3 is configured to connect an accessory shaft 4 to an instrument base 5 (not shown). The shaft connecting element 3 is configured as a hollow body 8 for passing through a transmission element guided in the accessory shaft 4, such as a pull rod (not shown). The shaft connecting element 3 has a first shape-coding dimension and a second shape-coding dimension. In the embodiment shown in FIG. 2, these are characterized, by way of example, by a diameter D and a length L.

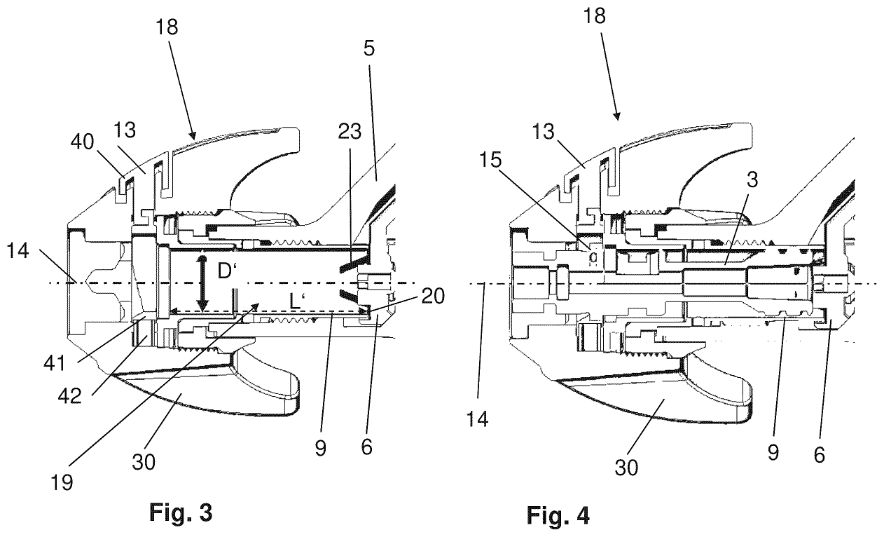

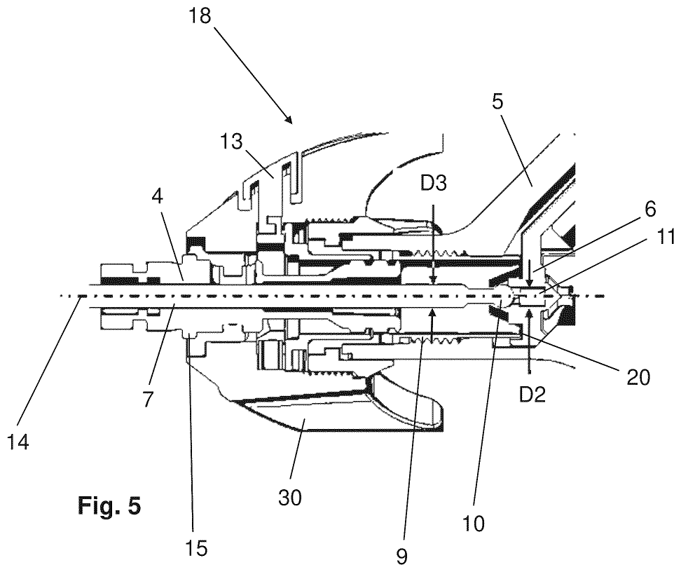

The first and the second shape-coding dimensions are dimensioned such that they correspond to a first and a second shape-coding dimension of a receiving device 18 of an instrument base 5, shown for example in FIG. 3, when the shaft connecting element 3 and the instrument base 5 belong to a common system and are provided so as to be couplable in an assembled state. If, on the other hand, the shaft connecting element 3 and the instrument base 5 do not correspond, they cannot be inserted into each other and cannot be coupled. The coupling or fixation between the shaft connecting element 3 and the instrument base 5 or the receiving device 18 can be carried out, for example, with a projection 15 on the shaft connecting element 3; this is shown in detail in FIGS. 5 to 8.

As can be seen in FIG. 1, there is a cover 37 with a so-called 4×90° geometry on the accessory shaft. This means that four recesses are arranged along the circumference of the cover, which can engage with a counter-contour on a rotary wheel assembly 30, shown in FIG. 3 et seq. and FIG. 16.

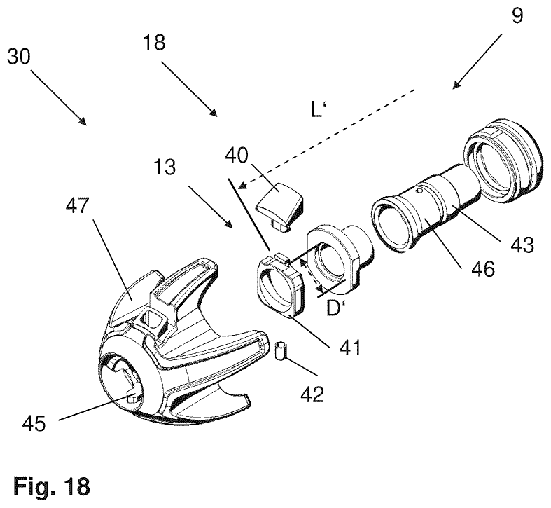

FIG. 3 shows an embodiment of a receiving device 18. The receiving device 18 is arranged on a shaft opening 19 of an instrument base 5 and has a first and second shape-coding dimension. A detailed illustration of the shape codings is shown, for example, in FIG. 18. A first shape-coding dimension can be, for example, a smallest inner diameter D′ and a second shape-coding dimension can be a provided shortest distance L′ between a latching means 13 and a stop 20. Consequently, for a corresponding connection, the dimensions D and L must match or correspond to the dimensions D′ and L′, such that a shaft connecting element 3 with an accessory shaft 4 can be assembled in an instrument base 5.

FIG. 4 shows the receiving device from FIG. 3 with the shaft connecting element 3 slid in.

The latching means 13 is configured as a spring-loaded latch which is integrated into the rotary wheel assembly 30, and which is configured to engage with the shaft connecting element 3. For example, the latch has a through-opening through which the shaft connecting element 3 can be passed. An outer head section 40 of the latch is provided as an actuating section, while a spring 42 preloading the latch is arranged on a foot section 41.

It can be seen that the shaft connecting element 3 is too long for the receiving device 18, such that the latching means 13 cannot latch with the projection 15. It is therefore not possible to secure the shaft connecting element 3 in the longitudinal direction 14 of the surgical instrument. A connection between the shaft connecting element 3 and the receiving device 18 is therefore not possible in this embodiment. This may be the case, for example, if mismatching elements of non-energized and energized surgical instruments are to be connected by mistake.

FIG. 5 shows the receiving device from FIG. 4 with a slid-in shaft connecting element 3 and pull rod 7. The pull rod 7 touches the through-opening 11, but cannot penetrate it. Consequently, a diameter D2 of the through-opening 11 is a third shape-coding dimension of the receiving device 18, and a diameter D3 of the pull rod 7, which is configured as a transmission element for electrical contacting, is a third shape-coding dimension of the shaft connecting element 3.

The diameter of the transmission element, i.e. in this case the diameter D3 of the pull rod 7 or the pull rod head 10, can be, for example, between 2 mm and 2.5 mm. The diameter D2 of the through-opening 11 can therefore be, for example, between 2.1 mm and 2.3 mm, such that a pull rod head 10 with a larger diameter cannot be guided through the through-opening 11. In this way, coding can be provided for different instrument systems. Advantageously, the excessively large pull rod head 10 is the first to touch the stop 20 or the excessively small through-opening 11, such that a user can detect an incorrect combination very early on due to a protruding shaft connecting element 3. In particular, damage caused by a with too much force can therefore be avoided.

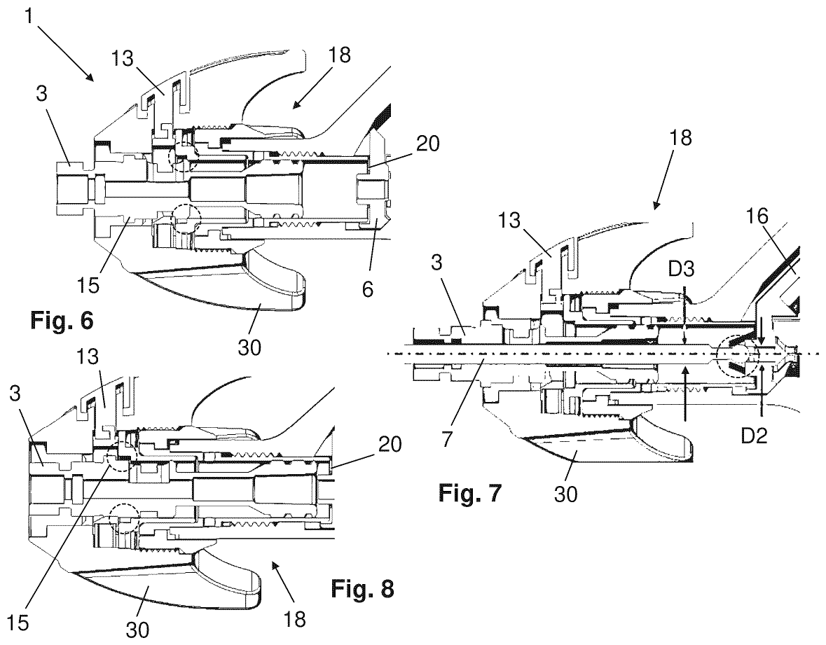

FIG. 6 also shows a connecting device 1 with non-matching elements. The diameter of the shaft connecting element 3 is greater than a diameter of the receiving device 18 and therefore cannot engage with the diameter of the receiving device 18. For example, the diameter may be approximately 0.1 mm to 1 mm greater, preferably 0.1 mm to 0.5 mm, preferably 0.1 mm to 0.3 mm, for example 0.2 mm. In this case, the diameter of the shaft connecting element 3 could be, for example, 8 mm and thus not engage with the diameter of the receiving device 18 with a dimension of 7.8 mm. The blocking regions are shown with dashed circles.

FIG. 7 shows another connecting device 1 with non-matching elements. In this case, the diameter of the pull rod head 10 is too large for the diameter of the through-opening 11. This corresponds to the case shown in FIG. 5.

FIG. 8 shows another connecting device 1 with non-matching elements. The shaft connecting element 3 can, for example, be at least 1 mm, in particular 1 mm to 5 mm, preferably 1 mm to 3 mm, for example 2 mm longer than the distance between the stop 20 and the latching means 13 of the receiving device 18. In this case, it can, for example, have a length of 26 mm, wherein the distance between the stop 20 and the latching means 13 of the receiving device 18 is only 24 mm. Consequently, the shaft connecting element 3 cannot be fixed in a latching manner in the receiving device 18.

Each of the embodiments of FIGS. 6 to 8 represent different shape-coding dimensions. For example, FIG. 6 shows a first shape-coding dimension and FIG. 8 shows a second shape-coding dimension. The situation in FIG. 7 may represent a third shape-coding dimension.

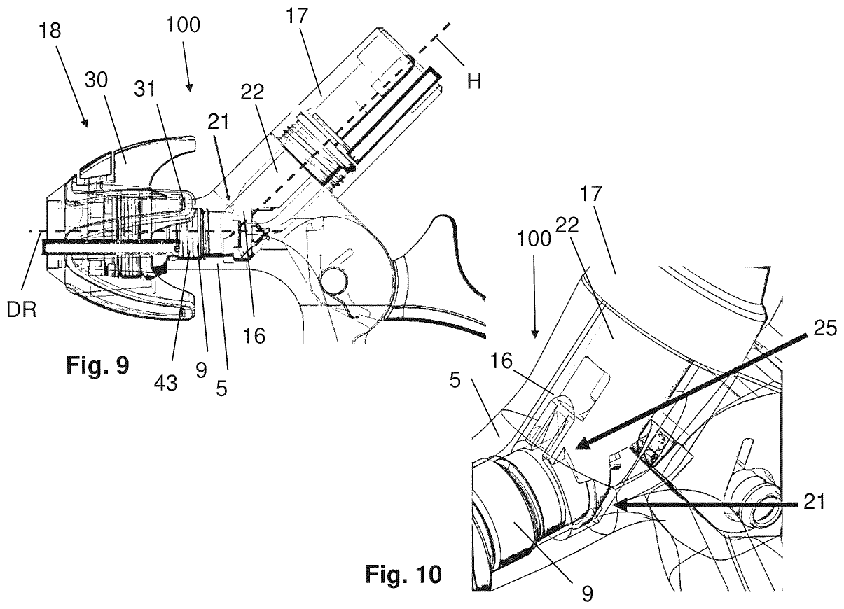

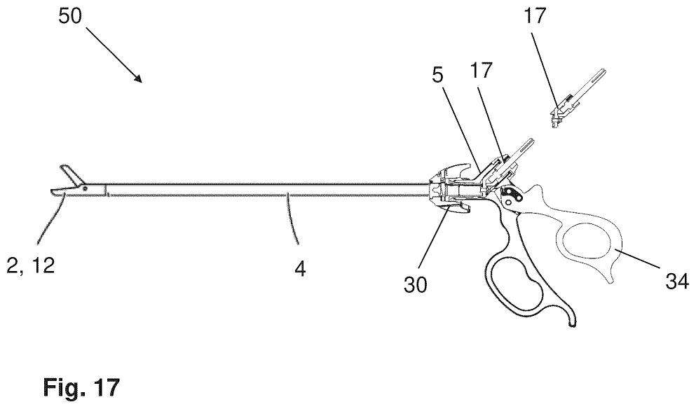

FIG. 9 and FIG. 10 show an embodiment of a connection assembly 100. The instrument base 5 is configured to control and/or operate a high-frequency tool 2, for example shown in FIG. 17.

A high-frequency connection 17 assembled in the instrument base has a connection element 16. The connection element 16 has an orientation section 22 which serves for the predetermined orientation of the HF connection 17 on a connection axis H within the instrument base 5. Furthermore, the connection element has a blocking section 21 for connection to a rotary wheel assembly 30 of the surgical instrument at a predetermined angle of a rotary wheel axis DR to the connection axis H. The rotary wheel assembly 30 has a coupling section 31 for coupling to the connection element 16. By means of the coupling process, the rotary wheel assembly 30 can be oriented in the predetermined rotary wheel axis DR with respect to the connection axis H in the instrument base 5.

For the rotational orientation of the connection element 16 and the rotary wheel assembly 30, at least one engagement element 25 shown in FIG. 10 is arranged in the instrument base 5, and can be connected to the coupling element 31 in an engaging or coupling manner.

The receiving device 18 can be in pre-assembled form. For example, the rotary wheel assembly 30 can be manufactured pre-assembled with corresponding receiving elements 9, which in particular have the shape-coding dimensions, as a specially coded embodiment. During assembly, the rotary wheel assembly 30 and the HF connection 17 can be slid into the instrument base 5 from different directions. The predetermined axes H and DR or their predetermined angles allow coupling without visibility.

By means of a blocking geometry 27, the two assemblies can finally be connected to one another in the instrument base 5 without any further aids. At the same time, the HF connection 17 and the receiving device 18, i.e. the rotary wheel assembly 30, can be oriented without any aids. The orientation ensures that the HF contacts are oriented concentrically with the inserted accessory shaft 4, the axis of which is defined by the rotary wheel assembly 30. To a certain extent, tolerance compensation of the components can also be achieved.

Advantageously, the axes H and DR are always consistent and arranged at a predefined angle to each other to ensure proper functioning of the surgical instrument. In particular, this avoids incorrect positioning, which could lead to contact problems during power transmission or to dragging or even blocking of the pull rod 7.

For assembly, for example, the rotary wheel assembly 30 can first be partially, in particular minimally, screwed into the instrument base 5 via a thread 43. The HF connection 17 can then be slid into the instrument base 5. Subsequently, the rotary wheel assembly 30 is advantageously screwed completely into the instrument base 5. When the HF connection 17 comes into contact with the receiving device 18 (with the rotary wheel assembly 30), they are oriented relative to each other. Since HF contact elements 23 are pre-assembled on the HF connection 17, any incorrect axial positioning that may occur can be tolerated or compensated for. Pre-assembly still allows complex and elaborate designs to be implemented, which can nevertheless be assembled comparatively easily due to the easy accessibility and/or can be configured with a comparatively small number of connection points.

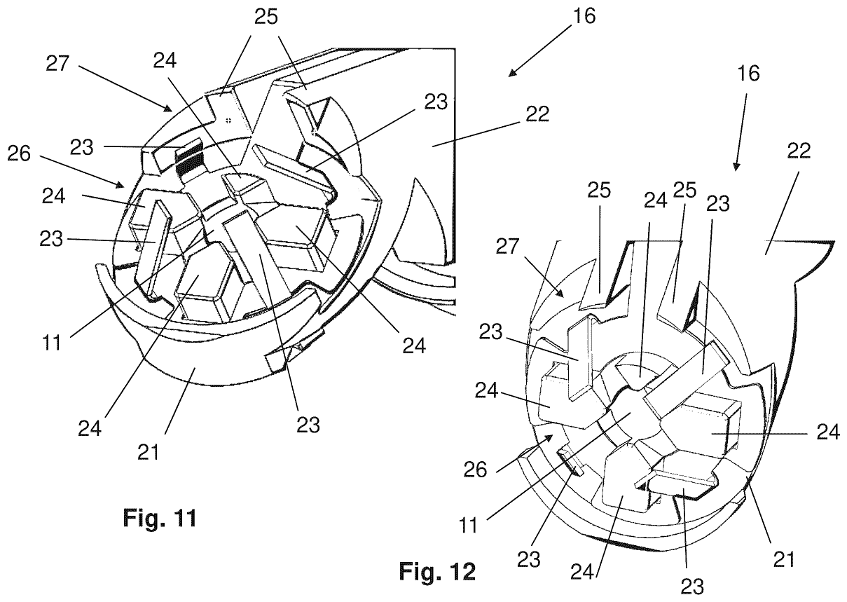

FIG. 11 and FIG. 12 show an embodiment of a connection element 16 of an HF connection 17. The connection element 16 has a blocking section 21 and an orientation section 22. In the region of the blocking section 21, a protective geometry 26 is provided to protect the HF contact assembly. Consequently, the protective geometry 26 can protect the HF contact elements 23 from damage when inserting the HF tool 2, in particular when inserting the shaft and/or the pull rod

The protective geometry 26 can be formed by a plurality of orientation projections 24, wherein the orientation projections 24 and the HF contact elements 23 are arranged adjacent to and alternately with one another. Consequently, the contact elements 23 are each placed in a kind of gap between the orientation projections 24. For example, if an incompatible accessory shaft with an incompatible pull rod head 10 is inserted into the instrument base 5, it will contact the surfaces of the orientation projections 24, thereby avoiding damage to the HF contact elements 23. Furthermore, it is possible for the HF contact elements 23 to spring into the gaps between the orientation projections 24 in the event of an overload, such that they are not destroyed or rendered unusable by plastic deformation. Furthermore, the protective geometry 26 can ensure the orientation and position of the HF contact elements 24.

The orientation projections 24, together with the engagement element 25, further form a blocking geometry 27 which is configured for rotationally secure engagement with the rotary wheel assembly 30. In particular, the receiving device 18 can have not only a rotary wheel assembly 30, but also at least one receiving element 9, which can also come into contact with the blocking geometry 27 and have a fixing effect. Such a receiving element 9 can be configured in several parts and is shown, for example, in FIG. 18 in an exemplary embodiment.

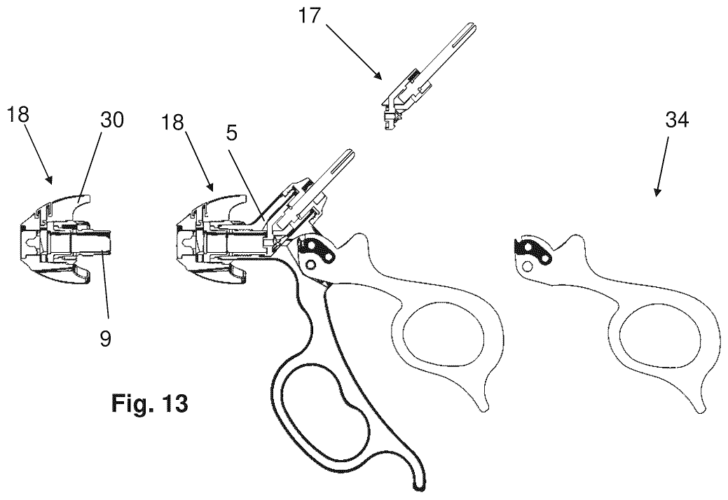

FIG. 13 shows an embodiment of an instrument base 5 with an HF connection 17, wherein the assemblies mounted with the instrument base 5 are additionally shown individually.

The HF connection 17 is configured as a pre-assemblable insert module for modular installation in and removal from the instrument base 5. For this purpose, the HF contact elements 23 are also pre-assembled on the insert module. The insert module can thus be inserted and assembled into the instrument base 5 along a connection axis H.

Furthermore, a removable thumb ring 34 is arranged on the instrument base.

The rotary wheel assembly is assembled or screwed in along the rotation axis DR.

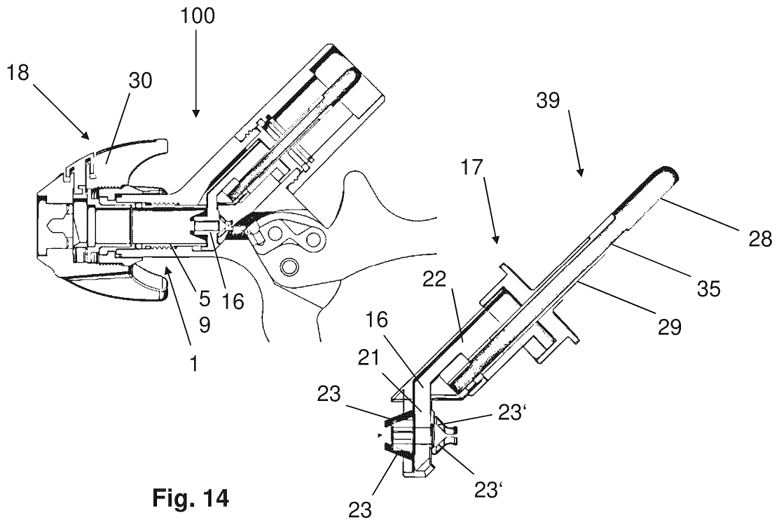

FIG. 14 shows a detailed view of the high-frequency connection 17, firstly in the assembled position and secondly isolated. The connection element 16 has a plug section 39 which can be connected in the direction of the connection axis H and to which the HF contact assembly is conductively coupled. The plug section 39 can have at least one plug pole 28 and an HF contact element 23 which is made from a continuous bent sheet metal part.

In the region of the through-opening 11, in the illustration on a side facing away from the blocking section 21, further HF contact elements 23′ are arranged, which serve to contact the pull rod 7. One HF contact element 23 and one HF contact element 23′ can each be made from a continuous bent sheet metal part. The plug section 39 can further have a solid steel core in the interior, which, for example, forms a second plug pole 29 and is welded to the HF contact element 23′.

The receiving device 18 and the HF connection 17 can be slid into the instrument base 5 from different sides, for example as shown in FIG. 13. Both modules are configured in such a way that they are functional with a minimal number of components and can be modularly assembled or exchanged. For example, the steel core protects against damage and is therefore particularly robust during use. Advantageously, the contact resistances within the HF connection 17 are produced by an integral connection, for example by welding, and are therefore very low-resistance. The components 17 and 18 can therefore be pre-assembled completely independently and then inserted and used directly in the instrument base 5 in the form of a “plug-and-play” assembly.

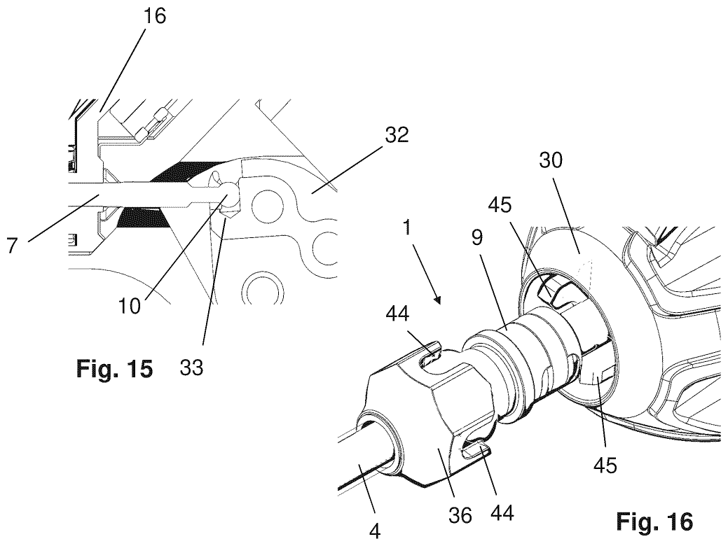

FIG. 15 shows an embodiment of a coupling of the pull rod head 10 with an actuating part of the instrument base 5. The pull rod 7 with the pull rod head 10 is passed through the through-opening 11 in the connection element 16 and guided to a coupling region 33 of the actuating element, which is configured here as a thumb ring 34. The thumb ring 34 forms a movable handle leg and has a ball holder in the coupling region 33. This ball holder can be integrated in a form-fitting manner during the manufacture of the thumb ring 34, in particular by an injection molding process. The pull rod head 10 can be received in this ball holder and thus transmit a movement, in particular for opening and closing the accessory insert 12, such as a scissor tool, from proximal to distal.

FIG. 16 shows an embodiment of a connecting device 1 in a detailed view.

As explained with reference to FIG. 1, there is a cover 37 with a so-called 4×90° geometry on the accessory shaft. The four recesses 44 provided for this purpose along the circumference of the cover 37 can engage with the counter-contour on the rotary wheel assembly 30. The counter-contour on the rotary wheel assembly 30 is formed by protruding pins 45 corresponding to the recesses 44; two of four pins 45 can be seen in the view.

FIG. 17 shows an embodiment of a surgical instrument 50.

The surgical instrument 50 has an accessory insert 12 in the form of an HF tool 2 at the distal end. The accessory shaft 7 runs from the distal end proximally to the instrument base 5. There, the accessory shaft 4 is received in the instrument base 5 as a receiving device via the rotary wheel assembly 30.

Furthermore, an HF connection 17 is plugged into the instrument base 5 in a different axis. The HF connection 17 is mounted at an angle of 45° on the top of the instrument base 5 and thus leads the high-frequency cable away from the operating field.

Furthermore, an actuating element, in this case the thumb ring 34, is assembled on the side of the instrument base 5 opposite the accessory shaft 4. When the actuating element is positioned horizontally, i.e. in this case when the thumb ring 34 is positioned horizontally, the accessory shaft can be decoupled from the actuating element, in particular the pull rod head can then be removed from the ball head holder.

The rotary wheel assembly 30 has the latching means 13 already explained. When the actuating element is positioned horizontally, pressing a button on the head section 41 of the latching means 13 is sufficient to separate the accessory shaft 4 from the actuating element. Then, for disassembly, the rotary wheel assembly 30, in this case by unscrewing, and the HF connection 17, in this case by pulling out, can also be removed modularly from the instrument base 5.

FIG. 18 shows a further embodiment of a connecting device 1 with a detailed view of the receiving device 18.

In this embodiment, the receiving device 18 of the rotary wheel assembly 30 has a receiving element 9 which is formed in several parts. Thus, the receiving device 18 can be formed from several sleeve-shaped elements which are fixed together. Individual elements can rotate within the instrument base 5 when assembled, while others can be fixed to it. In particular, the sleeve-shaped element 46 shown in the middle, which has the greatest extent in the longitudinal direction 14, can be screwed to the instrument base 5 via the thread 43. The other sleeve-shaped elements rotate when the rotary wheel of the rotary wheel assembly 30 is adjusted.

On the left side of the illustration, the rotary wheel 47 and the latching means 13 of the rotary wheel assembly 30 can also be seen. In the exploded view shown, the latching means 13 is shown detached from the rotary wheel 47.

In the distal opening of the rotary wheel assembly 30, the pin elements 45 integrated therein can be seen, which can engage in the recesses 44 of the cover 36, shown in FIG. 1.

In this embodiment, the first and second shape-coding dimensions are formed by the plurality of elements in the assembled state. These are schematically represented by a maximum diameter D′ and a maximum length L′indicated by dashed lines and naturally adjusted in the assembled state.

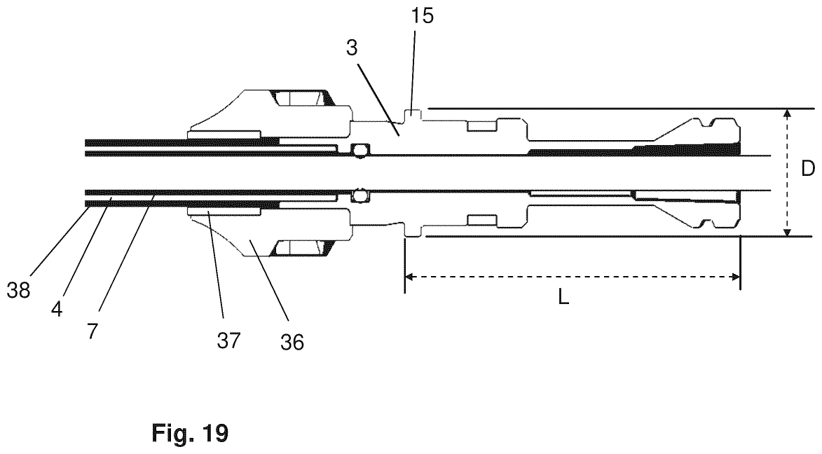

FIG. 19 shows an embodiment of a shaft connecting element 3 arranged on an accessory shaft 4.

In the connecting device shown here, the pull rod 7 runs in the accessory shaft 4. An insulating coating 38 is provided on the outside of the accessory shaft 4, for example a Halar coating (ECTFE).

A seal 37 is provided between the accessory shaft 4 and the cover 36, for example in the form of a sealing lip. On the shaft connecting element 3, the projection 15 can be seen, which is a measure of the first and second shape-coding dimensions. These are characterized by the diameter D and the length L.

The seal 37, the cover 36 and the shaft connecting element 3 can form a two-component insert injection molded part.

Although the present invention has been fully described above with reference to preferred exemplary embodiments, it is not limited thereto, but can be modified in a variety of ways. In particular, the shaft connecting element 3 can have a geometry that differs from that shown. Furthermore, the receiving device 18 can have a geometry that differs from the embodiment shown, in particular through differently shaped individual elements, such as individually shaped receiving elements 9. Likewise, the coupling between the rotary wheel assembly 30 and the cover 36 can be configured differently.

While specific embodiments of the invention have been shown and described in detail to illustrate the application of the principles of the invention, it will be understood that the invention may be embodied otherwise without departing from such principles.

### LIST OF REFERENCE CHARACTERS

1 Connecting device
2 High-frequency tool
3 Shaft connecting element
4 Accessory shaft
5 Instrument base
6 Guide element
7 Pull rod
8 Hollow body
9 Receiving element
10 Pull rod head
11 Through-opening
12 Accessory insert
13 Latching means
14 Longitudinal direction
15 Projection
16 Connection element
17 High-frequency connection
18 Receiving device
19 Shaft opening
20 Stop
21 Blocking section
22 Orientation section
23 High-frequency contact element
24 Orientation projection
25 Engagement element
26 Protective geometry
27 Blocking geometry
28 Plug pole
29 Second plug pole
30 Rotary wheel assembly
31 Coupling section
32 Handle
33 Coupling region
34 Thumb ring
35 Insulation element
36 Cover
37 Seal
38 Coating
39 Plug section
40 Head section
41 Foot section
42 Spring
43 Thread
44 Recess
45 Pin
46 Sleeve-shaped element
47 Rotary wheel
50 Surgical instrument
100 Connection assembly
D Diameter
D2 Diameter
D3 Diameter
L Length
DR Rotary wheel axis

1. A connection assembly for a surgical instrument or in a surgical instrument, the connection assembly comprising:
an instrument base that is configured to control and/or operate a high-frequency tool of the surgical instrument,
a high-frequency connection that comprises a connection element with a blocking section and an orientation section for a predetermined orientation of a connection axis of the high-frequency connection within the instrument base, and
a rotary wheel assembly comprising a coupling section for coupling to the blocking section of the high-frequency connection, such that a rotary wheel axis of the rotary wheel assembly can be oriented at a predetermined angle relative to the connection axis via a coupling process.

2. The connection assembly according to claim 1, wherein the blocking section forms a receptacle for the coupling section, which receptacle is concentric with the rotary wheel axis.

3. The connection assembly according to claim 1, wherein the connection element has in a region of the blocking section a through-opening which is configured to guide a pull rod of the high-frequency tool, wherein the rotary wheel assembly is configured such that the pull rod can be passed through the rotary wheel assembly.

4. The connection assembly according to claim 1, wherein the instrument base is configured as a connecting device to be connected to a manual guide element configured as a handle, to a manipulator coupling or to a robot holder.

5. The connection assembly according to claim 1, wherein the connection element has, in a region of the blocking section, a protective geometry for protecting a high-frequency contact assembly, which is configured to protect at least one high-frequency contact element from damage when inserting the high-frequency tool into the instrument base.

6. The connection assembly according to claim 1, wherein the blocking section has a blocking geometry which is configured for rotationally secure engagement with the coupling section of the rotary wheel assembly.

7. The connection assembly according to claims 5, wherein the blocking section has a blocking geometry which is configured for rotationally secure engagement with the coupling section of the rotary wheel assembly and the protective geometry (26) and the blocking geometry are formed integrally with one another.

8. The connection assembly according to claim 5, wherein the blocking section has a blocking geometry which is configured for rotationally secure engagement with the coupling section of the rotary wheel assembly and the blocking geometry and/or the protective geometry (26) has at least one orientation projection, wherein the at least one orientation projection and the high-frequency contact assembly are arranged adjacent to one another.

9. The connection assembly according to claim 8, wherein the high-frequency contact assembly has two or more high-frequency contact elements pre-assembled on the blocking section, wherein the orientation projections and the high-frequency contact elements are arranged next to one another, on the blocking section, such that the contact elements are each arranged in a gap between the orientation projections.

10. The connection assembly according to claim 1, wherein for the rotational orientation of the connection element and the rotary wheel assembly in the instrument base, at least one engagement element is arranged on the connection element, at a transition from the blocking section to the orientation section, and is configured to be contacted with a corresponding coupling element of the rotary wheel assembly and/or the instrument base.

11. A method for producing a connection assembly for a surgical instrument, the connection assembly comprising: an instrument base that is configured to control and/or operate a high-frequency tool of the surgical instrument; a high-frequency connection that comprises a connection element with a blocking section and an orientation section for a predetermined orientation of a connection axis of the high-frequency connection within the instrument base; and a rotary wheel assembly comprising a coupling section for coupling to the blocking section of the high-frequency connection, such that a rotary wheel axis of the rotary wheel assembly can be oriented at a predetermined angle relative to the connection axis, the method comprising the steps of:
attaching the rotary wheel assembly to a shaft opening of the instrument base, wherein the coupling section of the rotary wheel assembly is inserted into the instrument base;
sliding the high-frequency connection as, a pre-assembled high-frequency connection into a connection slot of the instrument base;
establishing contact between the coupling section and the blocking section of the high-frequency connection; and
completely inserting and fastening the coupling section in the instrument base for coupling to the blocking section.

12. The method according to claim 11, wherein for fastening, the coupling section is screwed into the instrument base, wherein the attachment of the rotary wheel assembly comprises securing a position by partial screwing-in.

13. The method according to claim 11, wherein the insertion of the coupling section into the shaft opening and the sliding of the high-frequency connection into the connection slot are carried out from different directions, such that the coupling of the coupling section to the blocking section comprises joining without visibility.

14. The method according to claim 13, wherein the connection between the connection element and the coupling element (9) of the rotary wheel assembly (18) is configured as a plug-and-play connection, wherein the surgical instrument can be used directly after the connection assembly has been made.

15. A surgical instrument, comprising:
an accessory comprising an accessory shaft and a high-frequency transmission element; and
a connection assembly, the connection assembly comprising: an instrument base that is configured to control and/or operate a high-frequency tool of the surgical instrument; a high-frequency connection that comprises a connection element with a blocking section and an orientation section for a predetermined orientation of a connection axis of the high-frequency connection within the instrument base; and a rotary wheel assembly comprising a coupling section for coupling to the blocking section of the high-frequency connection, such that a rotary wheel axis of the rotary wheel assembly can be oriented at a predetermined angle relative to the connection axis, wherein the accessory shaft is mechanically coupled to the rotary wheel assembly, and the high-frequency transmission element of the accessory.

16. The surgical instrument according to claim 15, wherein the accessory comprises a high-frequency forceps tool, and a pull rod guided in the accessory shaft is contacted with the high-frequency connection.

17. The connection assembly according to claim 6, wherein the protective geometry has at least one orientation projection, wherein the at least one orientation projection and the high-frequency contact assembly are arranged adjacent to one another.

18. The connection assembly according to claim 7, wherein the blocking geometry and/or the protective geometry has at least one orientation projection, wherein the at least one orientation projection and the high-frequency contact assembly are arranged adjacent to one another.
